(19) United States
(12) Patent Application Publication
(10) Pub. No.: US 2020/0299684 A1
(43) Pub. Date: Sep. 24, 2020

(54) SYSTEMS AND METHODS FOR POLYNUCLEOTIDE SCORING

(71) Applicant: TWIST BIOSCIENCE CORPORATION, San Francisco, CA (US)

(72) Inventors: Esteban TORO, Fremont, CA (US); James DIGGANS, San Carlos, CA (US); Siyuan CHEN, San Mateo, CA (US); Anthony COX, Mountain View, CA (US); Arthur VIGIL, San Francisco, CA (US); Kieran HERVOLD, San Francisco, CA (US)

(21) Appl. No.: 16/759,282

(22) PCT Filed: Oct. 26, 2018
(86) PCT No.: PCT/US2018/057857

§ 371 Date: Apr. 24, 2020

Related U.S. Application Data
(60) Provisional application No. 62/578,309, filed on Oct. 27, 2017.

(51) Int. Cl. C12N 15/10; G16B 30/20; G16B 30/10

(52) U.S. Cl. CPC C12N 15/1089; G16B 30/10; G16B 30/20

(57) ABSTRACT

The present disclosure describes software tools for predicting the feasibility of synthesizing and assembling polynucleotides. Polynucleotide scoring tools describe herein provide automated methods for predicting efficient strategies and reaction conditions for synthesizing and assembling polynucleotides.

### CROSS-REFERENCE

This application claims the benefit of U.S. provisional patent application No. 62/578,309 filed on Oct. 27, 2017, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 15, 2018, is named 44854-740_601_ SL.txt and is 968 bytes in size.

### BACKGROUND

Highly efficient chemical gene synthesis with high fidelity and low cost has a central role in biotechnology and medicine, and in basic biomedical research. De novo gene synthesis is a powerful tool for basic biological research and biotechnology applications. While various methods are known for the design and synthesis of relatively short fragments in a small scale, these techniques often suffer from predictability, scalability, automation, speed, accuracy, and cost.

### BRIEF SUMMARY

Provided herein are computerized systems for polynucleotide assembly comprising: a general purpose computer; and a computer readable medium comprising functional modules including instructions for the general purpose computer, wherein said computerized system is configured for operating in a method of: receiving operating instructions, wherein the operating instructions comprise a full length polynucleotide sequence; automatically generating a plurality of designs each comprising a plurality of polynucleotide sequences, wherein the plurality of polynucleotide sequences each comprises at least one overlap region of 30 to 50 bases in length, wherein each overlap region is complementary to another overlap region, and wherein each of the at least one overlap regions does not comprise a homopolymeric sequence; and automatically selecting a design from the plurality of designs that comprises polynucleotide sequences having the lowest variance in Tm between the at least one overlap regions. Further provided herein are computerized systems wherein assembly of the polynucleotide sequences having the lowest variance in Tm between the at least one overlap regions results in the full length polynucleotide sequence. Further provided herein are computerized systems further comprising splitting the full-length polynucleotide into two or more sub-fragments, and selecting a design for each of the sub-fragments, wherein each sub-fragment comprises at least one overlap region complementary to another sub-fragment, and assembly of the sub-fragments results in the full-length polynucleotide. Further provided herein are computerized systems wherein the full length polynucleotide sequence is at least 500 bases in length. Further provided herein are computerized systems wherein the full length polynucleotide sequence is at least 1000 bases in length. Further provided herein are computerized systems wherein the full length polynucleotide sequence is at least 2000 bases in length. Further provided herein are computerized systems wherein the full length polynucleotide sequence is at least 5,000 bases in length. Further provided herein are computerized systems wherein the full length polynucleotide sequence is at least 10,000 bases in length. Further provided herein are computerized systems wherein the at least one overlap regions comprise an average of 30 percent to 70 percent GC content. Further provided herein are computerized systems wherein the at least one overlap regions comprise an average of 40 percent to 60 percent GC content. Further provided herein are computerized systems wherein each of the at least one overlap regions comprises 30 percent to 70 percent GC content. Further provided herein are computerized systems wherein each of the at least one overlap regions comprises 40 percent to 60 percent GC content. Further provided herein are computerized systems wherein each of the at least one overlap regions is 20 to 40 bases in length. Further provided herein are computerized systems wherein the plurality of polynucleotide sequences comprises at least 5 polynucleotide sequences. Further provided herein are computerized systems wherein the plurality of polynucleotide sequences comprises at least 10 polynucleotide sequences. Further provided herein are computerized systems wherein the plurality of polynucleotide sequences comprises at least 50 polynucleotides. Further provided herein are computerized systems wherein the plurality of polynucleotide sequences comprises 25 to 50 polynucleotide sequences. Further provided herein are computerized systems wherein the plurality of polynucleotide sequences comprises 10 to 30 polynucleotide sequences. Further provided herein are computerized systems wherein each polynucleotide sequence is 40 to 200 bases in length. Further provided herein are computerized systems wherein each polynucleotide sequence is 50 to 150 bases in length. Further provided herein are computerized systems wherein the full length polynucleotide sequence encodes a cDNA sequence for a gene or gene fragment. Further provided herein are computerized systems for polynucleotide assembly comprising: a general purpose computer; and a computer readable medium comprising functional modules including instructions for the general purpose computer, wherein said computerized system is configured for operating in a method of: receiving operating instructions, wherein the operating instructions comprise a full length polynucleotide sequence; automatically generating a plurality of designs each comprising a plurality of polynucleotide sequences, wherein the plurality of polynucleotide sequences each comprises at least one overlap region of 30 to 50 bases in length, wherein each overlap region is complementary to another overlap region, wherein each of the at least one overlap regions does not comprise a homopolymeric sequence, and wherein assembly of the polynucleotide sequences from a design generates a long fragment, wherein assembly of a plurality of long fragments results in the full-length polynucleotide sequence; and automatically selecting a design from the plurality of designs that comprises polynucleotide sequences having the lowest variance in Tm between the at least one overlap regions.

Provided herein are methods for polynucleotide synthesis comprising: receiving operating instructions, wherein the operating instructions comprise a full length polynucleotide sequence; automatically generating a plurality of designs each comprising a plurality of polynucleotide sequences, wherein the plurality of polynucleotide sequences each comprises at least one overlap region of 30 to 50 bases in length, wherein each overlap region is complementary to another overlap region, and wherein each of the at least one overlap regions does not comprise a homopolymeric sequence; automatically selecting a design from the plurality of designs that comprises polynucleotide sequences having the lowest variance in Tm between the at least one overlap regions; and synthesizing the polynucleotides having the lowest variance in Tm between the at least one overlap regions. Further provided herein are methods further comprising assembling the full length polynucleotide sequence from the polynucleotides having the lowest variance in Tm between the at least one overlap regions. Further provided herein are methods further comprising splitting the full-length polynucleotide into two or more sub-fragments, and selecting a design to synthesize a plurality of polynucleotides for each of the sub-fragments, wherein assembly of the polynucleotides generates the sub-fragment, and wherein each sub-fragment comprises at least one overlap region complementary to a another sub-fragment, and assembly of the sub-fragments results in the full-length polynucleotide. Further provided herein are methods wherein the full length polynucleotide sequence is at least 500 bases in length. Further provided herein are methods wherein the full length polynucleotide sequence is at least 1000 bases in length. Further provided herein are methods wherein the full length polynucleotide sequence is at least 5,000 bases in length. Further provided herein are methods wherein the at least one overlap regions comprise an average of 30 percent to 70 percent GC content. Further provided herein are methods wherein in each of the at least one overlap regions comprises 30 percent to 70 percent GC content. Further provided herein are methods wherein the at least one overlap regions comprise an average of 40 percent to 60 percent GC content. Further provided herein are methods wherein in each of the at least one overlap regions comprises 40 percent to 60 percent GC content. Further provided herein are methods wherein each of the at least one overlap regions is 20 to 40 bases in length. Further provided herein are methods wherein each of the at least one overlap regions is 25 to 40 bases in length. Further provided herein are methods wherein the plurality of polynucleotide sequences comprises at least 5 polynucleotide sequences. Further provided herein are methods wherein the plurality of polynucleotide sequences comprises at least 50 polynucleotide sequences. Further provided herein are methods wherein the plurality of polynucleotide sequences comprises at least 10 polynucleotide sequences. Further provided herein are methods wherein each polynucleotide sequence is 40 to 200 bases in length. Further provided herein are methods wherein each polynucleotide sequence is 50 to 150 bases in length. Further provided herein are methods wherein the full length polynucleotide sequence encodes a cDNA sequence for a gene or gene fragment. Further provided herein are methods for polynucleotide synthesis comprising: receiving operating instructions, wherein the operating instructions comprise a full length polynucleotide sequence; automatically generating a plurality of designs each comprising a plurality of polynucleotide sequences, wherein the plurality of polynucleotide sequences each comprises at least one overlap region of 30 to 50 bases in length, wherein each overlap region is complementary to another overlap region, and wherein each of the at least one overlap regions does not comprise a homopolymeric sequence, wherein assembly of the polynucleotide sequences from a design generates a long fragment, wherein assembly of a plurality of long fragments results in the full-length polynucleotide sequence; automatically selecting a design that comprises polynucleotides having the lowest variance in Tm between the at least one overlap regions; and synthesizing the polynucleotides having the lowest variance in Tm between the at least one overlap regions.

Provided herein are computerized systems for polynucleotide assembly comprising: a general purpose computer; and a computer readable medium comprising functional modules including instructions for the general purpose computer, wherein said computerized system is configured for operating in a method of: receiving operating instructions, wherein the operating instructions comprise a full length polynucleotide sequence; automatically generating a plurality of designs each comprising a plurality of polynucleotide sequences; automatically generating a pass score for each of the polynucleotide sequences, wherein the pass rate score is determined by assigning a weighted value for one or more of: average percent GC content of the polynucleotide sequence; the percent GC content for a region of continuous bases in the polynucleotide sequence; length of the polynucleotide sequence; maximum melting temperature for direct repeats in the polynucleotide sequence; density of repeats in the polynucleotide sequence, wherein the density of repeats is a number of repeating bases divided by a total length of each polynucleotide sequence; and length of homopolymers in the polynucleotide sequence; and assigning a numerical value to at least one design for a number of clones to screen for the full length sequences following assembly, wherein the numerical value is assigned based on the pass rate score. Further provided herein are computerized systems further comprising splitting the full-length polynucleotide into two or more sub-fragments, and selecting a design for each of the sub-fragments, wherein each sub-fragment comprises at least one overlap region complementary to another sub-fragment, and assembly of the sub-fragments results in the full-length polynucleotide. Further provided herein are computerized systems wherein the pass rate score is determined by assigning a weighted value to the percent GC content for a region of continuous bases in the polynucleotide sequence, and wherein the region of continuous bases in the polynucleotide sequence is at least 25 bases in length. Further provided herein are computerized systems wherein the number of repeating bases is at least 6 bases. Further provided herein are computerized systems wherein the number of repeating bases is at least 6-15 bases. Further provided herein are computerized systems wherein the homopolymers each have a length of at least 10 bases. Further provided herein are computerized systems wherein the homopolymers each have a length of at least 6-15 bases. Further provided herein are computerized systems wherein the plurality of polynucleotide sequences comprises at least 30 polynucleotide sequences. Further provided herein are computerized systems wherein the plurality of polynucleotide sequences comprises 25-50 polynucleotide sequences. Further provided herein are computerized systems wherein the clones are generated by prokaryotic cells or eukaryotic cells. Further provided herein are computerized systems wherein the method further comprises rejecting a design that receives a numerical value less than a predetermined numerical value threshold, and wherein nucleic acids encoding for the polynucleotide sequences of the rejected design are not synthesized. Further provided herein are computerized systems wherein the method further comprises synthesizing nucleic acids encoding for the plurality of polynucleotide sequences from at least one design. Further provided herein are computerized systems wherein the method further comprises assembling the plurality of polynucleotides of at least one design into a nucleic acid encoding for the full-length polynucleotide sequence, wherein assembling comprising PCA. Further provided herein are computerized systems wherein the method further comprises transforming the nucleic acid encoding for the assembled full-length polynucleotide into at least one cell to generate at least one clone. Further provided herein are computerized systems wherein the method further comprises sequencing at least one clone to confirm assembly of the nucleic acid encoding for the correctly assembled full-length polynucleotide sequence. Further provided herein are computerized systems for polynucleotide assembly comprising: a general purpose computer; and a computer readable medium comprising functional modules including instructions for the general purpose computer, wherein said computerized system is configured for operating in a method of: receiving operating instructions, wherein the operating instructions comprise a full length polynucleotide sequence; automatically generating a plurality of designs each comprising a plurality of polynucleotide sequences, wherein assembly of the polynucleotide sequences from a design generates a long fragment, wherein assembly of a plurality of long fragments results in the full-length polynucleotide sequence; automatically generating a pass score for each of the polynucleotide sequences, wherein the pass rate score is determined by assigning a weighted value for one or more of: average percent GC content of the polynucleotide sequence; the percent GC content for a region of continuous bases in the polynucleotide sequence; length of the polynucleotide sequence; maximum melting temperature for direct repeats in the polynucleotide sequence; density of repeats in the polynucleotide sequence, wherein the density of repeats is a number of repeating bases divided by a total length of each polynucleotide sequence; and length of homopolymers in the polynucleotide sequence; and assigning a numerical value to at least one design for a number of clones to screen for the full length sequences following assembly, wherein the numerical value is assigned based on the pass rate score.

Provided herein are methods for polynucleotide synthesis comprising: receiving operating instructions, wherein the operating instructions comprise a full length polynucleotide sequence; automatically generating a plurality of designs each comprising a plurality of polynucleotide sequences; automatically generating a pass score for each the polynucleotide sequences, wherein the pass rate score is determined by assigning a weighted value for one or more of: average percent GC content of the polynucleotide sequence; the percent GC content for a region of continuous bases in the polynucleotide sequence; length of the polynucleotide sequence; maximum melting temperature for direct repeats in the polynucleotide sequence; density of repeats in the polynucleotide sequence, wherein the density of repeats is a number of repeating bases divided by a total length of the polynucleotide sequence; and length of homopolymers in the polynucleotide sequence; assigning a numerical value to at least one design for a number of clones to screen for the full length sequences following assembly, wherein the numerical value is assigned based on the pass rate score; and synthesizing polynucleotides having the pass score above a threshold value. Further provided herein are methods further comprising assembling the full length polynucleotide sequence from the polynucleotides having the pass score above a threshold value. Further provided herein are methods further comprising splitting the full-length polynucleotide into two or more sub-fragments, and selecting a design to synthesize a plurality of polynucleotides for each of the sub-fragments, wherein assembly of the polynucleotides generates the sub-fragment, and wherein each sub-fragment comprises at least one overlap region complementary to a another sub-fragment, and assembly of the sub-fragments results in the full-length polynucleotide. Further provided herein are methods wherein the pass rate score is determined by assigning a weighted value to the percent GC content for a region of continuous bases in the polynucleotide sequence, and wherein the region of continuous bases in the polynucleotide sequence is at least 25 bases in length. Further provided herein are methods wherein the number of repeating bases is at least 6 bases. Further provided herein are methods wherein the number of repeating bases is at least 6-15 bases. Further provided herein are methods wherein the homopolymers each have a length of at least 10 bases. Further provided herein are methods wherein the homopolymers each have a length of at least 6-15 bases. Further provided herein are methods wherein the plurality of polynucleotide sequences comprises at least 30 polynucleotide sequences. Further provided herein are methods wherein the plurality of polynucleotide sequences comprises 25-50 polynucleotide sequences. Further provided herein are methods wherein the clones are generated by prokaryotic cells or eukaryotic cells. Further provided herein are methods wherein the method further comprises rejecting a design that receives a numerical value less than a predetermined numerical value threshold, and wherein nucleic acids encoding for the polynucleotide sequences of the rejected design are not synthesized. Further provided herein are methods wherein the method further comprises synthesizing nucleic acids encoding for the plurality of polynucleotide sequences from at least one design. Further provided herein are methods wherein the method further comprises assembling the plurality of polynucleotides of at least one design into a nucleic acid encoding for the full-length polynucleotide, wherein assembling comprising PCA. Further provided herein are methods wherein the method further comprises transforming a nucleic acid encoding for the assembled full-length polynucleotide sequence into at least one cell to generate at least one clone. Further provided herein are methods wherein the method further comprises sequencing at least one clone to confirm assembly of the nucleic acids encoding for the full-length polynucleotide sequence. Further provided herein are methods for polynucleotide synthesis comprising: receiving operating instructions, wherein the operating instructions comprise a full length polynucleotide sequence; automatically generating a plurality of designs each comprising a plurality of polynucleotide sequences, wherein assembly of the polynucleotide sequences from a design generates a long fragment, wherein assembly of a plurality of long fragments results in the full-length polynucleotide sequence; automatically generating a pass score for the polynucleotide sequences, wherein the pass rate score is determined by assigning a weighted value for one or more of: average percent GC content of the polynucleotide sequence; the percent GC content for a region of continuous bases in the polynucleotide sequence; length of the polynucleotide sequence; maximum melting temperature for direct repeats in the polynucleotide sequence; density of repeats in the polynucleotide sequence, wherein the density of repeats is a number of repeating bases divided by a total length of the polynucleotide sequence; and length of homopolymers in the polynucleotide sequence; assigning a numerical value to at least one design for a number of clones to screen for full length sequences following assembly, wherein the numerical value is assigned based on the pass rate score; and synthesizing polynucleotides having the pass score above a threshold value. Further provided herein are methods further comprising assembling the full length polynucleotide sequence from the polynucleotides having the pass score above a threshold value. Further provided herein are methods further comprising splitting the full-length polynucleotide into two or more sub-fragments, and selecting a design to synthesize a plurality of polynucleotides for each of the sub-fragments, wherein assembly of the polynucleotides generates the sub-fragment, and wherein each sub-fragment comprises at least one overlap region complementary to a another sub-fragment, and assembly of the sub-fragments results in the full-length polynucleotide.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The technical features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of the following.

FIG. 1 illustrates an example of a program comprising modules for polynucleotide assembly design.

FIG. 2A illustrates an example of a polynucleotide assembly method.

FIG. 2B illustrates an example of an overlap region between two polynucleotides.

FIG. 3 illustrates an example output of assembly difficulty for various sequence parameters.

FIG. 4 illustrates a complex sequence represented by “g”s buried inside a polynucleotide, so that these sequences are outside overlap regions. FIG. 4 discloses SEQ ID NOS 1-3, respectively, in order of appearance.

FIG. 5 illustrates a design for assembly of a full length polynucleotide.

FIG. 6A illustrates a visualization for a filter map of run 1.

FIG. 6B illustrates a visualization for a filter map of run 2.

FIG. 7 illustrates a plot of synthesis pass rate verses calculated score.

FIG. 8 illustrates a computing system.

FIG. 9 illustrates a computer system.

FIG. 10 is a block diagram illustrating an architecture of a computer system.

FIG. 11 is a diagram demonstrating a network configured to incorporate a plurality of computer systems, a plurality of cell phones and personal data assistants, and Network Attached Storage (NAS).

FIG. 12 is a block diagram of a multiprocessor computer system using a shared virtual address memory space.

### DETAILED DESCRIPTION

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the polynucleotide being known and chosen before the synthesis or assembly of the nucleic acid molecules.

Provided herein are compositions, systems and methods for production of synthetic polynucleotides. The term oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA.

Libraries comprising synthetic genes may be constructed by a variety of methods described in further detail elsewhere herein, such as PCA (polymerase chain assembly), non-PCA gene assembly methods or hierarchical gene assembly, combining (“stitching”) two or more double-stranded polynucleotides to produce larger DNA units (i.e., a chassis). Libraries of large constructs may involve polynucleotides that are at least 1, 1.5, 2, 3, 4, 5, 6, 7, 8, 9, 10, 15, 20, 30, 40, 50, 60, 70, 80, 90, 100, 125, 150, 175, 200, 250, 300, 400, 500 kb long or longer. The large constructs can be bounded by an independently selected upper limit of about 5000, 10000, 20000 or 50000 base pairs. The synthesis of any number of polypeptide-segment encoding nucleotide sequences is described herein, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein, may comprise at least one region encoding for exon sequence(s) without an intervening intron sequence found in the corresponding genomic sequence. Alternatively, the corresponding genomic sequence to a cDNA may lack an intron sequence in the first place.

After assembly of polynucleotide fragments (e.g., from libraries, full length polynucleotides, etc.) described herein, such fragments may be cloned into host organisms. For example, assembled polynucleotides are inserted into vectors via restriction endonuclease/ligation, Gibson Assembly®, Golden Gate® Assembly, transposase-based ligation (e.g., Gateway® cloning) or other method for inserting a polynucleotide into a vector. In some instances, vectors are transformed into host organisms through electroporation, chemical means, or any other method of nucleic acid transformation. In some instances, polynucleotides are directly transformed into host organisms. Host organisms (“clones”) may then be analyzed to identify or sort correctly assembled polynucleotides. Often less than all clones created will comprise the correctly assembled sequence, therefore clones are analyzed to identify the correct sequence. For difficult assembly designs, a larger number of clones are in some cases analyzed. For example, host organisms with correctly assembled polynucleotides are identified by means of growth rate, an active reporter (e.g., fluorescence, beta-galactosidase, phosphoresce, resistance), or other means. In some instances, host organisms are sequenced to identify correctly assembled polynucleotides. In some instances, host organisms comprise eukaryotic or prokaryotic cells. In some instances, host organisms comprise bacteria or yeast.

Polynucleotide Design Schemes

Provided herein are compositions, methods and systems for the design and synthesis of nucleic acids (e.g., genes) involving the division of a nucleic acid sequence into a plurality of smaller polynucleotides, i.e. fragments of the longer nucleic acid, for de novo synthesis and subsequent assembly to form the nucleic acid of interest. Further provided herein are methods for the assessment and selection of optimal polynucleotides for the synthesis processes. As described herein, factors considered in the design process may include individual sequence specific features (e.g., annealing temperature, overhang length, GC and AT content, and nucleobase repeat region) or a hierarchical feature of the collective plurality of polynucleotides (e.g., non-specific binding to other polynucleotides in the population to be synthesized, avoidance of large repeat sequences at a terminus of any individual polynucleotide, and schemes for breaking very long nucleic acids into intermediate assembly schemes prior to complete assembly). Further provided herein are methods for generating assembly designs based on predetermined assembly conditions, scoring assembly designs for difficulty, and selecting optimal designs for synthesis. As described herein, factors considered in selecting an optimal design may include the categories of PCR assembly conditions (temperatures, polymerase, additives, etc.), empirical data from prior assemblies, off-target homology relationships between polynucleotide fragments, overlap annealing temperature uniformity, and the presence/location of complex sequences in the design. Evaluation of sequences in a given design may comprise scoring of fragments, sub-sequences, or full-length sequences.

Provided herein are methods to generate assembly designs for the generation of full length polynucleotide sequence from assembly of de novo synthesized shorter polynucleotide sequences. These designs may comprise full length sequences, assembly conditions or instructions, sequences of fragments of the full length sequence, a score representing the difficulty of the assembly, or other information relevant to the assembly of full length polynucleotides. The methods may create designs based on preset parameters. The different steps in a method may proceed automatically without further user input, and optionally direct the automatic synthesis of the full length sequence using the assembly design. A plurality of smaller designs may together comprise a larger design for a given full length polynucleotide sequence. The size of full length sequences may be at least 500, 1000, 2000, 5000, 10,000, or at least 20,000 bases in length.

Methods described herein may comprise a series of steps that are used for considering the results of a previous step, and generating a new result. The result of a previous step may be used for decision making in a subsequent step. Larger steps may comprise a series of smaller steps; for example, after receiving design parameters for polynucleotide fragment assembly and a full length polynucleotide sequence of a given length to be assembled, one or more designs comprising a list of smaller polynucleotide sequences capable of assembly into the full length sequence is generated. In some instances, steps include generating visual representations of outputs, such as assembly designs or filters. In some instances, steps generating lists of sequences, sequence fragments, design rankings, assembly parameters, or other output consistent with polynucleotide design or assembly are utilized.

Steps in the methods described herein comprise variables for analysis, such as one or more sequences. Steps may also comprise consideration of polynucleotide design categories, each providing data on minimum and maximum Tm, overlap length, non-overlap length, GC % of overlaps, or parameters specific to terminal assembly fragments (those on the 5′ or 3′ ends of the full length sequence).

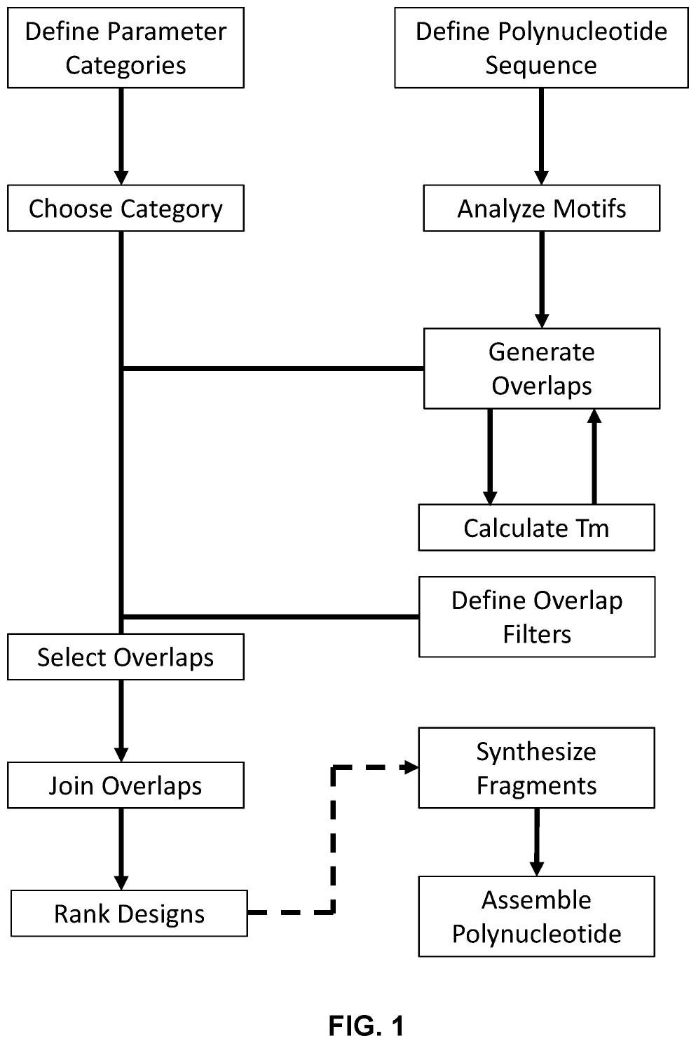

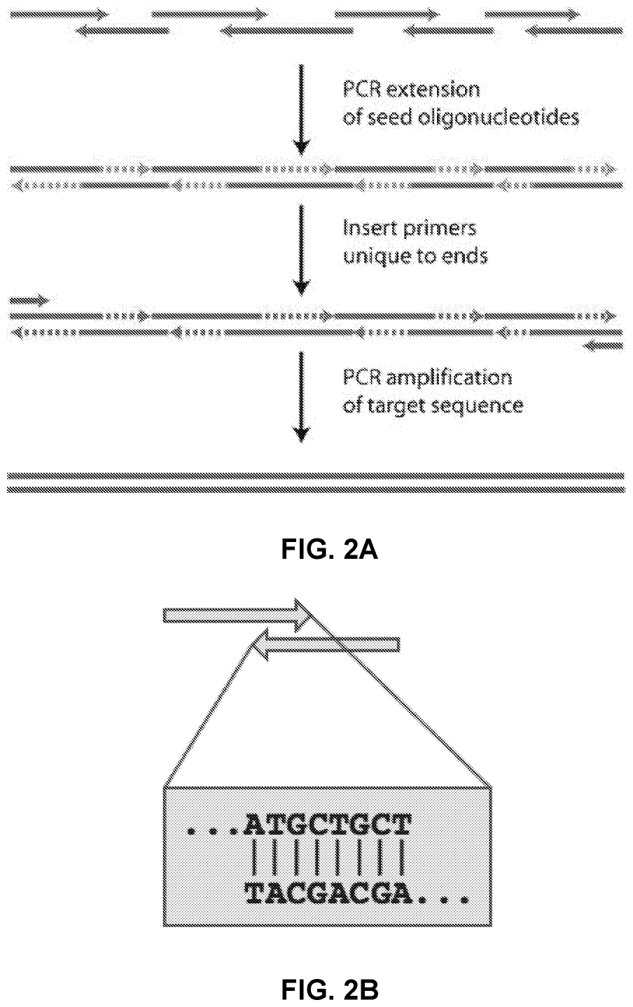

In a first scheme, a polynucleotide designer comprises steps of: analyzing motifs in a full length polynucleotide sequence, generating overlaps, choosing a category, selecting overlaps, calculating Tm, joining overlaps, and ranking designs. Optionally, the fragments from a design are synthesized and assembled into the full length polynucleotide. A non-limiting exemplary arrangement of steps for this process is illustrated in FIG. 1. In one instance, assembly of fragments is conducted using overlap PCR (FIG. 2A). Overlap regions are regions of the fragments that comprise one or more complementary bases, designed to anneal together during assembly. For example, a fragment comprises an overlap region on the 5′ terminus, and an overlap on the 3′ terminus. Alternately, a fragment may comprise an overlap region on only the 5′ terminus or only on the 3′ terminus. An exemplary overlap between two fragments is illustrated in FIG. 2B. In some instances, one or more bases in the overlap region are not complementary. Methods described herein may comprise any number of fragments for assembly of the full length polynucleotide. For example, an assembly (or assembly design) comprises at least 5, 10, 20, 30, 40, 50, 60, 70, or more than 70 fragments. In some instances, an assembly comprises at least 30 fragments. In some instances, an assembly comprises at least 50 fragments. In some instances, an assembly comprises 25-50 fragments. Consistent with the specification, a polynucleotide designer comprises additional steps that facilitate the design and/or assembly of full length sequences. Consistent with the specification, steps may be omitted or reordered as needed in the methods described herein.

In one step, a sequence is evaluated to determine if the sequence comprises any complex sequence regions. Non-limiting examples of complex sequences are hairpins, loops, high or low % GC content, repeating sequences, repeating bases (homopolymers), homomultimers, (ability of sequence to self-multimerize), palindromic sequences, or any other sequence property that could potentially interfere with correct hybridization during assembly. In some instances, high GC content is no less than 60% GC, 70%, 80%, 90%, or greater than 90% GC. In some instances, low GC content is no more than 40% GC, 30%, 20%, 10%, or less than 10% GC. The location of complex sequences is then considered for overlap selection.

In another step, a set of overlapping fragments which are capable of assembly into a full length sequence is generated from the full length sequence and are a predetermined range of acceptable overlap lengths. The set of overlapping fragments is then used for overlap selection. Overlapping fragments meeting the desired Tm criteria are generated by calculating Tm of the overlap regions with a Tm calculator algorithm. The Tm of the overlap is the melting temperature at which a strand and its complementary strand separate. Various algorithms and methods for calculating Tm are well known to those skilled in the art, including but not limited to the Marmur formula, Wallace formula, Breslauer method, Schildkraut salt correction formula, SantaLucia method, or any other Tm calculating algorithm or method. In some instances, BioPython is used to calculate Tm. In some instances, complex sequence regions are buried inside of fragments to avoid the complex sequence region from being part of an overlap region (FIG. 4).

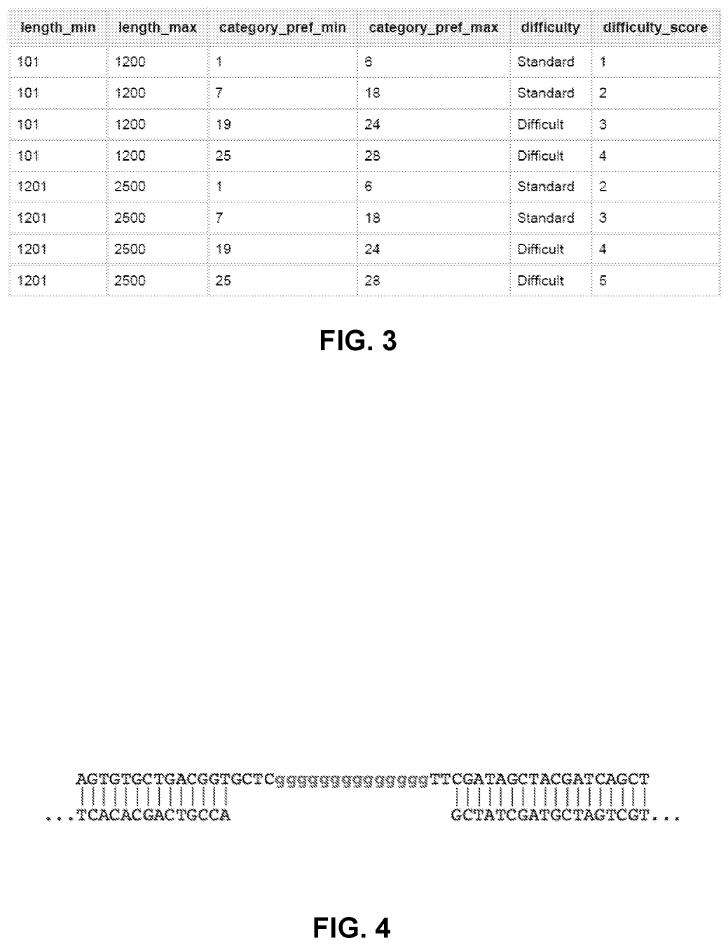

In yet another step, a category comprising empirical sequence parameters for the assembly of sequence fragments is chosen. For example, a first category comprises assembly instructions for a high GC sequence. Potential designs may be generated from the first category, and then a new category is chosen to search additional designs. The choice of category in some instances is considered for overlap selection. In some instances, different categories are further sorted into bins based on common parameters. Category parameters include but are not limited to assembly difficulty, extension and annealing temperatures, salt concentrations, additive concentrations, fragment lengths, location of complex sequences, enzymes, extension and annealing times or other variable affecting assembly conditions. In some instances, the order in which categories are populated with designs is automatically determined based on the full length sequence. In some instances, full length sequences can be assigned categories, which are used to predict the difficulty of assembly (FIG. 3.)

In an additional step, overlaps are selected based on motif analysis, generated overlaps, and categories to generate a list of overlaps that meet the design parameters of the overlap joining step. Overlap selections often are determined by overlap filters, which are used to generate designs conforming to design parameters. Exemplary design parameters include but are not limited to overlap Tm, location of complex sequence regions, overlap length, GC content, or other design parameter than can affect assembly of overlapping fragments.

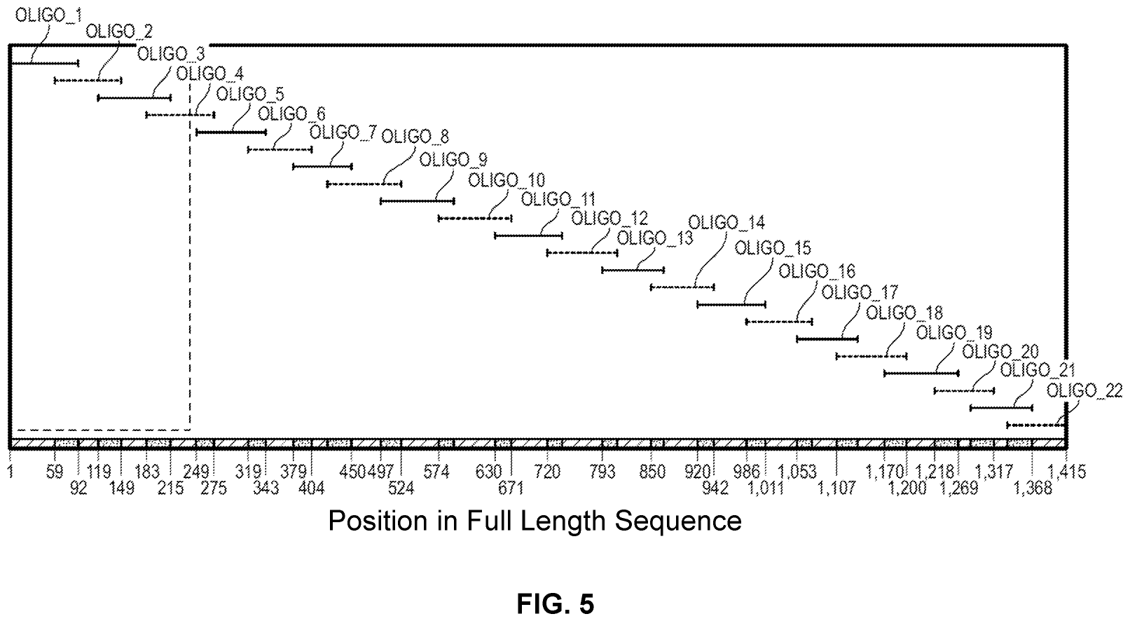

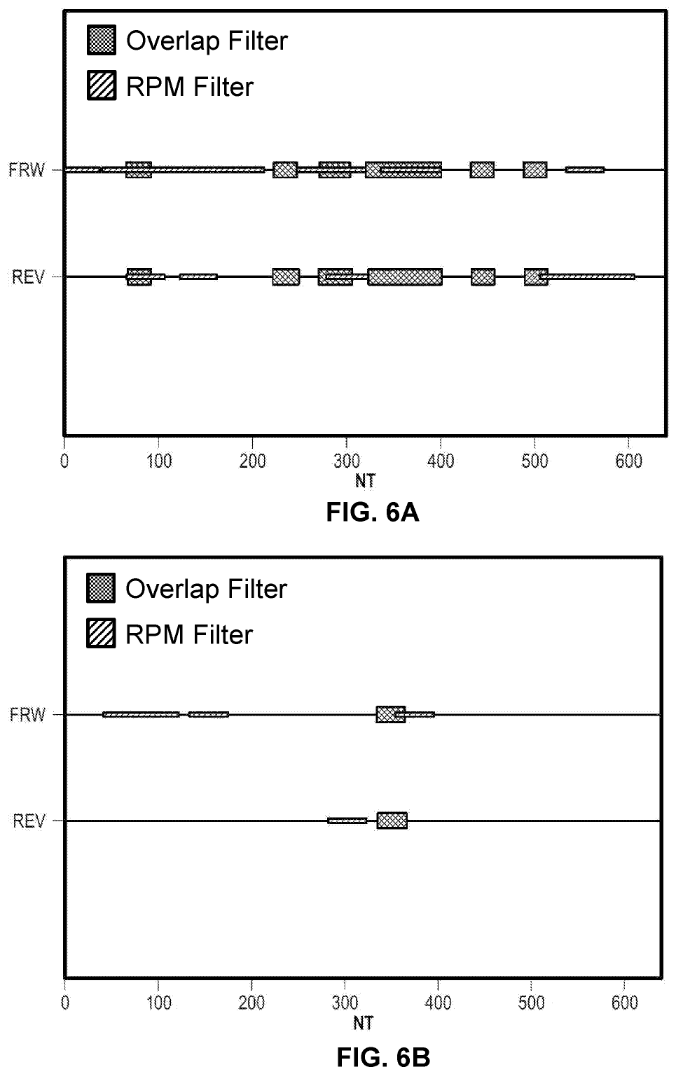

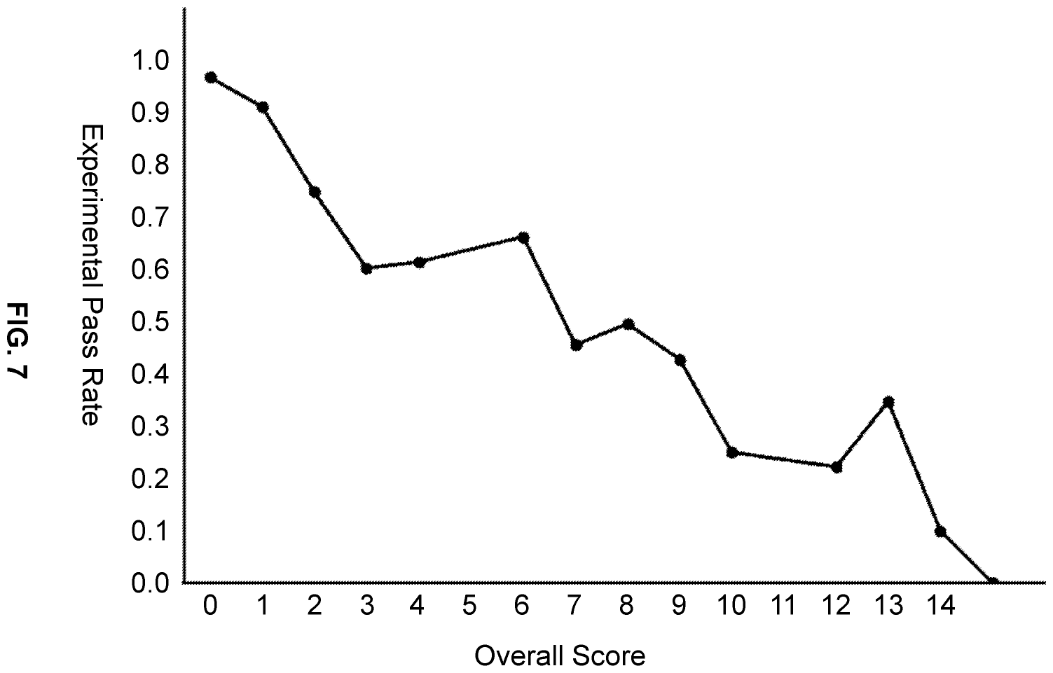

In another step, fragment sequences comprising overlaps are assembled into a design for the full length sequence. In one example, a graph is generated wherein the nodes of the graph are overlaps, and an edge is created between two nodes if the implied fragment has a length meeting the design criteria. A path through the graph is then identified, which corresponds to a design. In some aspects, fragments corresponding to the regions near the 5′ and/or 3′ regions of the full length sequence are longer or shorter than the interior fragments. In some instances, uncorrelated designs that maximize overlap diversity are generated. In some instances, a graphical visualization of the design, showing the organization of overlapping fragments is generated. An exemplary visualization of a design is illustrated in FIG. 5. In some instances, designs are influenced by one or more filters. For example, an exemplary filter that controls the number of non-complementary bases in an overlap region as depicted in FIGS. 6A-6B for forward (FRD) and reverse (REV) fragment polynucleotides designed to assemble a 640 bp sequence. Shaded boxes represent sequence locations in the sequence filtered out for use in overlap regions using a specific set of filtering variables or conditions for both overlap (evaluation of overlap Tm) and RPM filters. Thicker boxes (on the Y-axis) in FIGS. 6A-6B indicate sequence regions filtered out for use as overlap regions due to the overlap filter (i.e. under the conditions chosen for the filter, the Tm is outside the chosen range for assembly). Thinner boxes (on the Y-axis) in FIGS. 6A-6B indicate sequences filtered out for use as overlap regions due to the RPM filter (i.e. sequence in these regions contain direct repeats or palindromic sequence outside the chosen range for assembly assembly). In some instances, the RPM filter checks for repeating sequencings on the same strand (direct repeats). The exemplary design in FIG. 6A requires at least 7 matching bases on the 3′ end of the fragment, and at least 19 matches in any position of the overlap. The exemplary design in FIG. 6B requires at least 8 matching bases on the 3′ end of the fragment, and at least 20 matches in any position of the overlap. The number of bases for an overlap region in some instances is 10 to 50 bases in length. The number of bases for an overlap region in some instances is 10 to 30 bases in length. The number of bases for an overlap region in some instances is 20 to 40 bases in length. Designs optionally comprise any specific requirements for the overlap region, and are not limited by the examples disclosed herein.

In another step, a series of designs for a given category are ranked and scored (or assigned a numerical value) based on a set of parameters. Such scores may be used to adjust fragment synthesis parameters, assembly conditions, or cloning methods and/or colony sampling. Such parameters are in some instances assigned a weighted value and used to generate a (pass) score for a design. Exemplary parameters for fragments, sub-sequences, or full-length sequences include the average percent GC content, the percent GC content for a region of continuous bases in the sequence (e.g., a “window”), length of the sequence, variance of fragment overlap Tm (hybridized to its reverse complement), maximum melting temperature for direct repeats in the sequence, density of repeats in the sequence (for example, repeat length divided by the total length of the sequence), and length of homopolymers. Scoring may also be conducted on fragments or sub-sequences, in order to select designs. In some instances, the parameters comprise the standard deviation (or variance) of fragment overlap Tm, for example providing a favorable ranking to a design with a smaller standard deviation (or variance) of overall fragment overlap Tm. In some instances, overlap Tm is measured between an overlap region and its reverse complement. In another example, a favorable ranking is given to a design with fragments that are less homologous to other distal fragments in the design, thus preventing incorrect cross-hybridization during assembly. In some instances the parameters comprise diversity of overlap design. In some instances, statistics and decision trees describing how each design was generated or ranked is generated. In some instances, the three highest scoring designs are generated. In some instances, the top scoring design is automatically executed by synthesizing the overlapping fragments. In some instances, the synthesized fragments are automatically assembled into a full length polynucleotide.

Characteristics of overlap regions (such as Tm, GC content, repeats, or other factor) may be used to score or evaluate designs. In some instances, designs comprising overlaps with homopolymeric sequences are rejected. In yet another example, the percent GC content of the overlaps imparts a favorable score. In some instances, an average GC content of 30% to 70% in polynucleotide overlaps of a design is favorable to selection of the design. In yet another example, the percent GC content of the overlaps imparts a favorable score. In some instances, an average GC content of 40% to 60% in polynucleotide overlaps of a design is favorable to selection of the design. In yet another example, the percent GC content of the overlaps imparts a favorable score. In some instances, a GC content of 30% to 70% in each polynucleotide overlap of a design is favorable to selection of the design. In yet another example, the percent GC content of the overlaps imparts a favorable score. In some instances, a GC content of 40% to 60% in each polynucleotide overlap of a design is favorable to selection of the design. In another example, the GC content may be analyzed for a given region of continuous bases in a sequence. In some instances, a region of about 25, 50, 75, or about 100 bases is analyzed for percent GC content.

Further provided herein are methods to generate assembly designs for a full length polynucleotide sequence wherein a longer full length sequence is divided in smaller sub-sequences. For example, a hierarchical assembly (HA) method generates two or more smaller sub-sequences from the larger full length sequence, generates individual designs for each sub-sequence, wherein the sub-sequences can be subsequently assembled into the larger full length polynucleotide. In some instances split points are chosen in a similar manner as an overlap selection step (e.g., meeting design criteria such as minimizing complex sequencing regions, desired overlap Tm, etc.). Potential split points that comprise complex sequence regions are rejected, and alternate split points are evaluated until the regions adjacent to the split point meet one or more design criteria. The size of the full length sequence may determine if the sequence should be split into smaller sequences. In some instances, a full length sequence greater than 2.1 kb is split. In some instances, a full length sequence greater than 1 kb, 2 kb, 3 kb, 5 kb, 10 kb, or more than 10 kb is split. In some instances, the splitting process continues until sub-sequences of a desirable size are obtained, and the sub-sequences are each subjected to a design method. In some instances, the full length polynucleotide is split into no more than 2, 3, 4, 5, 6, 7, 8, 9, 10, 20, 50, 75, 100, 200, 500, 1,000, or no more than 5,000 sub-sequences. In some instances, the full length polynucleotide is split into about 2, 3, 4, 5, 6, 7, 8, 9, 10, 20, 50, 75, 100, 200, 500, or 1,000 sub-sequences. In some instances, the desired sub-sequence size is less than 0.5 kb, 1 kb, 1.5 kb, 2 kb, 3 kb, 5 kb, or less than 10 kb.

Further provided herein are methods to generate assembly designs for full length polynucleotide sequences wherein the full length sequences are evaluated before designs are created in order to reject full length sequences or assembly fragments from designs that are likely to be difficult to synthesize. For example, a difficult overall full length sequence could be sorted into complex and simple sequence regions. For example, a full length sequence with overall GC content greater than 65%, or greater than 30%, 40%, 50%, 60%, or greater than 75% is rejected. In some instances, a full length sequence with overall GC content greater than 65% and less than 30% is rejected. In some instances, a full length sequence with overall GC content greater than 55% and less than 35% is rejected. In some instances, a full length sequence with overall GC content greater than 50% and less than 40% is rejected. In some instances, a sequence having a window (or region of consecutive bases) in a sequence with a GC content less than 30% or greater than 70% is rejected. In another example, a full length sequence with an exact repeat of 25 bases or greater separated by at least 100 bases is rejected. In some instances, a full length sequence with an exact repeat of 25 consecutive bases or greater is rejected. In some instances, a full length sequence with an exact repeat of 20 consecutive bases or greater is rejected. In another example, a full length sequence with an exact repeat of at least 5, 10, 20, 25, 30, 35, 40 or more than 40 bases separated by at least 100 bases, or at least 10, 20, 50, 75, 100, 150, or at least 200 bases is rejected. In another example, a full length sequence with an exact repeat with a Tm of greater than 64° C., greater than 60° C., 65° C., 70° C., 75° C., or greater than 80° C. is rejected. In some instances, complex sequence regions are identified and optionally visualized on the full length sequence. Full length sequences may be subjected to a hierarchical assembly (HA) method described herein, with additional modifications to provide a rapid assembly design. For example, the full length sequence is divided into sub-sequences with a predetermined maximum length, and each sub-sequence is subjected to a design method.

Computer Algorithms for Polynucleotide Synthesis

Provided herein are computer algorithms to generate assembly designs or instructions for the assembly of full length polynucleotide sequences. These designs may comprise full length sequences, assembly conditions or instructions, sequences of fragments of the full length sequence, a score representing the difficulty of the assembly, or other information relevant to the assembly of full length polynucleotides. A plurality of smaller designs may together comprise a larger design for a given full length polynucleotide sequence. The computer algorithms may create designs based on preset parameters. The different algorithms may proceed automatically without further user input, and optionally direct the automatic synthesis of the full length sequence using the assembly design. Designs may be represented visually for user analysis in some instances. Further provided herein are computer algorithms that comprise a series of modules for processing input data, and generating an output. The output may be an input for a subsequent module. Larger modules may comprise a series of smaller modules. For example, a module receives input parameters for polynucleotide fragment assembly and a full length polynucleotide sequence of a given length to be assembled, and outputs one or more design instructions comprising a list of smaller polynucleotide sequences (fragments) capable of assembly into the full length sequence. In some instances, modules generate visual representations of outputs, such as assembly designs or filters. In some instances, modules generate outputs comprising lists of sequences, sequence fragments, design rankings, assembly parameters, or other output consistent with polynucleotide design or assembly. Consistent with the specification, modules may be omitted or reordered as needed in the methods described herein. Fragments may refer to polynucleotides that are capable of assembly into larger polynucleotides, such as sub-fragments, long fragments or full-length fragments. A plurality of sub-fragments or long fragments are assembled, for example, into a full-length polynucleotide. A full-length polynucleotide sequence is in some instances divided into a plurality of shorter fragment polynucleotides (sub-fragments, long fragments) to facilitate assembly. These shorter fragments are in some instances further divided into even shorter fragments. This process may be continued interactively until polynucleotide sequences of the smallest desired size are reached.

Module inputs or outputs may comprise variables for analysis, such as one or more sequences. By way of non-limiting example, sequences may be stored in FASTA, FASTQ, EMBL, GCG, Genbank, IG, Genomatix, or any other format that allows storage of sequence data. Module inputs or outputs may also comprise polynucleotide design categories each providing data on minimum and maximum Tm, overlap length, non-overlap length, GC % of overlaps, or parameters specific to terminal assembly fragments (those on the 5′ or 3′ ends of the full length sequence). In one example, module inputs or outputs are stored in a JSON file, but other data files capable of storing module inputs or outputs are also used. In some instances, an input or output comprises a summary of the workflow used to generate one or more designs.

In a first algorithm, a polynucleotide designer comprises modules: a motif analyzer, an overlap generator, a category chooser, an overlap selector, a Tm calculator, an overlap joiner, a design ranker, and an overlap filter. Consistent with the specification, a polynucleotide designer in some instances comprises additional modules that facilitate the design and assembly of full length sequences. In some instances, modules are arranged in series or in parallel. In some instances, one or more modules are omitted from the algorithm.

In a first module, a motif analyzer receives an input sequence, and determines if the sequence comprises any complex sequence regions. Non-limiting examples of complex sequences are hairpins, loops, high or low % GC content, repeating sequences, repeating bases, palindromic sequences, or any other sequence property that could potentially interfere with correct hybridization during assembly. In some instances, high GC content is no less than 60% GC, 70%, 80%, 90%, or greater than 90% GC. In some instances, low GC content is no more than 40% GC, 30%, 20%, 10%, or less than 10% GC. The location of complex sequences is then used as input for an overlap selector module. Alternately or in combination, regions of the full length sequence comprising complex sequences are annotated.

In a second module, an overlap generator receives input of a full length sequence, and the desired range of lengths for the overlaps. A set of candidate overlap regions is then generated, a subset of which will define polynucleotides capable of assembly into the full length sequence and are a predetermined range of acceptable overlap lengths. Overlaps meeting the desired Tm criteria are generated by calculating Tm of overlap regions with a Tm estimation algorithm. The Tm of an overlap is the temperature at which one half the molecules of a strand and its complementary strand separate. Various algorithms and methods for calculating Tm are well known to those skilled in the art, including but not limited to the Marmur formula, Wallace formula, Breslauer method or other Tm calculating algorithm or method. In some instances, these algorithms and methods are used alone or in combination with a salt correction method. For example, salt correction methods include but are not limited to the Schildkraut salt correction formula, SantaLucia method, Owczarzy method, or any other salt correcting algorithm or method. In some instances, the SantaLucia method comprises the nearest-neighbor method. In some instances, BioPython is used to calculate Tm. In some instances, complex sequence regions are buried inside of fragments to avoid the complex sequence region from being part of an overlap region. The set of overlapping fragments is then used as input for the overlap selector.

In a third module, a category chooser receives input comprising empirical sequence parameters for the assembly of sequence fragments. For example, a first category comprises assembly instructions for a high GC sequence. Potential designs may be generated from the first category, and then a new category is chosen to search additional designs. The category chooser outputs a category to the overlap selector. In some instances, different categories are further sorted into bins based on common parameters. Category parameters include but are not limited to assembly difficulty, extension and annealing temperatures, salt concentrations, additive concentrations, fragment lengths, location of complex sequences, enzymes, extension and annealing times or other variable affecting assembly conditions. In some instances, the order in which categories are populated with designs is automatically determined based on the full length sequence. In some instances, full length sequences can be assigned categories, which are used to predict the difficulty of assembly.

In a fourth module, an overlap selector receives input from the motif analyzer, overlap generator, and category chooser modules, and outputs a list of overlaps that meet the design parameters to the overlap joiner module. Overlap selections often are determined by input from overlap filters, which are used to generate designs conforming to design parameter inputs. Exemplary design parameter inputs include but are not limited to overlap Tm, location of complex sequence regions, overlap length, GC content, or other design parameter input than can affect the correct assembly of overlapping fragments.

In a fifth module, an overlap joiner receives input from the overlap selector module comprising overlap sequences. The overlap joiner module then assembles fragments comprising the overlaps, and generates a design. In one example, the overlap joiner module generates a graph wherein the nodes of the graph are overlaps, and an edge is created between two nodes if the implied fragment has a length meeting the design criteria. The overlap joiner module then identifies a path through the graph, which corresponds to a design. In some aspects, fragments corresponding to the regions near the 5′ and/or 3′ regions of the full length sequence are longer or shorter than the interior fragments. In some instances, the overlap joiner module generates uncorrelated designs that maximize overlap diversity. In some instances, the overlap joiner module generates a graphical visualization of the design, showing the organization of overlapping fragments.

In a sixth module, a design ranker receives a series of designs for a given category, and scores the designs based on a set of parameters. In some instances, the parameters comprise the standard deviation of fragment overlap Tm, for example providing a favorable ranking to a design with a smaller standard deviation of overall fragment overlap Tm. In another example, a favorable ranking is given to a design with fragments that are less homologous to other distal fragments in the design, thus preventing incorrect cross-hybridization during assembly. In some instances the parameters comprise diversity of overlap design. In some instances, the design ranker module outputs statistics and decision trees describing how each design was generated or ranked. In some instances, the design ranker module outputs the three highest scoring designs. In some instances, the top scoring design is automatically executed via a polynucleotide synthesis device to synthesize the fragments. In some instances, the synthesized fragments are automatically assembled into a full length polynucleotide.

Further provided herein are algorithms to generate assembly designs for a full length polynucleotide sequence wherein a longer full length sequence is divided in smaller sub-sequences. For example in a second algorithm, a hierarchical assembly (HA) module receives a full length sequence as input, and outputs two or more smaller sub-sequences that are inputted into a polynucleotide designer algorithm, as it may be advantageous to split larger full length sequences into smaller sequences which can be synthesized and subsequently assembled. In some instances, individual designs for each sub-sequence are generated, wherein the sub-sequences can be subsequently assembled into the larger full length polynucleotide. In some instances the HA module chooses split points are chosen in a similar manner as the overlap selector module (e.g., meeting design criteria such as minimizing complex sequencing regions, desired overlap Tm, etc.). Potential split points that comprise complex sequence regions are rejected, and alternate split points are evaluated until the regions adjacent to the split point meet one or more design criteria. The size of the full length sequence may determine if the sequence should be split into smaller sequences. In some instances, a full length sequence greater than 2.1 kb is split by the HA module. In some instances, a full length sequence greater than 1 kb, 2 kb, 3 kb, 5 kb, 10 kb, or more than 10 kb is split by the HA module. In some instances, the splitting process continues until full length fragments of a desirable size are obtained, and the sub-sequences are each subjected to a polynucleotide design algorithm. In some instances, the full length polynucleotide is split into no more than 2, 3, 4, 5, 6, 7, 8, 9, 10, 20, 50, 75, 100, 200, 500, 1,000, or no more than 5,000 sub-sequences. In some instances, the full length polynucleotide is split into about 2, 3, 4, 5, 6, 7, 8, 9, 10, 20, 50, 75, 100, 200, 500, or 1,000 sub-sequences. In some instances, the desired full length fragment size is less than 0.5 kb, 1 kb, 1.5 kb, 2 kb, 3 kb, 5 kb, or less than 10 kb. Algorithms are used to identify candidate split points of larger full length fragments. For example, a random walk algorithm is used to identify candidate split points. In some instances, candidate split points are identified using a gradient descent algorithm. In some instances, candidate split points are identified using a genetic algorithm. Further provided herein are algorithms to generate assembly designs for full length polynucleotide sequences rapidly. In a third algorithm, full length sequences are evaluated before designs are created in order to reject full length sequences that are likely to be difficult to synthesize. For example, a difficult overall full length sequence could be sorted into complex and simple sequence regions. For example, a full length sequence with overall GC content greater than 65%, or greater than 30%, 40%, 50%, 60%, or greater than 75% is rejected. In another example, a full length sequence with an exact repeat of 25 bases or greater separated by at least 100 bases is rejected. In another example, a full length sequence with an exact repeat of at least 5, 10, 20, 25, 30, 35, 40 or more than 40 bases separated by at least 100 bases, or at least 10, 20, 50, 75, 100, 150, or at least 200 bases is rejected. In another example, a full length sequence with an exact repeat with a Tm of greater than 64° C., greater than 60° C., 65° C., 70° C., 75° C., or greater than 80° C. is rejected. In some instances, complex sequence regions are identified and optionally visualized on the full length sequence. Full length sequences may be subjected to a hierarchical assembly (HA) module, with additional modifications to provide a rapid assembly design. For example, the full length sequence is divided into sub-sequences with a predetermined maximum length, and each sub-sequence is subjected to a design algorithm.

Polynucleotides

The full length sequence length may vary depending on the application. In some instances, the full length sequence length is 100 bases to 100,000 bases. In some instances, the full length sequence length is at least 100 bases. In some instances, the full length sequence length is at most 100,000 bases. In some instances, the full length sequence length is 100 bases to 200 bases, 100 bases to 500 bases, 100 bases to 1,000 bases, 100 bases to 2,000 bases, 100 bases to 5,000 bases, 100 bases to 10,000 bases, 100 bases to 20,000 bases, 100 bases to 50,000 bases, 100 bases to 100,000 bases, 200 bases to 500 bases, 200 bases to 1,000 bases, 200 bases to 2,000 bases, 200 bases to 5,000 bases, 200 bases to 10,000 bases, 200 bases to 20,000 bases, 200 bases to 50,000 bases, 200 bases to 100,000 bases, 500 bases to 1,000 bases, 500 bases to 2,000 bases, 500 bases to 5,000 bases, 500 bases to 10,000 bases, 500 bases to 20,000 bases, 500 bases to 50,000 bases, 500 bases to 100,000 bases, 1,000 bases to 2,000 bases, 1,000 bases to 5,000 bases, 1,000 bases to 10,000 bases, 1,000 bases to 20,000 bases, 1,000 bases to 50,000 bases, 1,000 bases to 100,000 bases, 2,000 bases to 5,000 bases, 2,000 bases to 10,000 bases, 2,000 bases to 20,000 bases, 2,000 bases to 50,000 bases, 2,000 bases to 100,000 bases, 5,000 bases to 10,000 bases, 5,000 bases to 20,000 bases, 5,000 bases to 50,000 bases, 5,000 bases to 100,000 bases, 10,000 bases to 20,000 bases, 10,000 bases to 50,000 bases, 10,000 bases to 100,000 bases, 20,000 bases to 50,000 bases, 20,000 bases to 100,000 bases, or 50,000 bases to 100,000 bases. In some instances, the full length sequence length is about 100 bases, about 200 bases, about 500 bases, about 1,000 bases, about 2,000 bases, about 5,000 bases, about 10,000 bases, about 20,000 bases, about 50,000 bases, or about 100,000 bases. In some instances, the full length sequence length is more than 100,000 bases.

In some instances, the overlap length is about 5 bases to about 200 bases. In some instances, the overlap length is at least about 5 bases. In some instances, the overlap length is at most about 200 bases. In some instances, the overlap length is about 5 bases to about 10 bases, about 5 bases to about 20 bases, about 5 bases to about 40 bases, about 5 bases to about 100 bases, about 5 bases to about 200 bases, about 10 bases to about 20 bases, about 10 bases to about 40 bases, about 10 bases to about 100 bases, about 10 bases to about 200 bases, about 20 bases to about 40 bases, about 20 bases to about 100 bases, about 20 bases to about 200 bases, about 40 bases to about 100 bases, about 40 bases to about 200 bases, or about 100 bases to about 200 bases. In some instances, the overlap length is about 5 bases, about 10 bases, about 20 bases, about 40 bases, about 100 bases, or about 200 bases.

In some instances, the overall fragment length (including the overlap regions) is about 5 bases to about 1,000 bases. In some instances, the overall fragment length is at least about 5 bases. In some instances, the overall fragment length is at most about 1,000 bases. In some instances, the overall fragment length is about 5 bases to about 10 bases, about 5 bases to about 20 bases, about 5 bases to about 40 bases, about 5 bases to about 100 bases, about 5 bases to about 200 bases, about 5 bases to about 1,000 bases, about 10 bases to about 20 bases, about 10 bases to about 40 bases, about 10 bases to about 100 bases, about 10 bases to about 200 bases, about 10 bases to about 1,000 bases, about 20 bases to about 40 bases, about 20 bases to about 100 bases, about 20 bases to about 200 bases, about 20 bases to about 1,000 bases, about 40 bases to about 100 bases, about 40 bases to about 200 bases, about 40 bases to about 1,000 bases, about 100 bases to about 200 bases, about 100 bases to about 1,000 bases, or about 200 bases to about 1,000 bases. In some instances, the overall fragment length is about 5 bases, about 10 bases, about 20 bases, about 40 bases, about 100 bases, about 200 bases, or about 1,000 bases. In some instances, the overall fragment length is greater than 1000 bases. In some instances, the overall fragment length is about 30 to about 200 bases in length. In some instances, the overall fragment length is about 30 to about 150 bases in length. In some instances, the overall fragment length is about 40 to about 200 bases in length. In some instances, the overall fragment length is about 50 to about 200 bases in length. In some instances, the overall fragment length is about 50 to about 150 bases in length.

Digital Processing Device

The platforms, systems, media, and methods described herein may include a digital processing device, or use of the same. In some examples, the digital processing device may include one or more hardware central processing units (CPUs) or general purpose graphics processing units (GPGPUs) that carry out the device's functions. In some examples, the digital processing device may further comprise an operating system configured to perform executable instructions. The digital processing device may be optionally connected a computer network. The digital processing device may be optionally connected to the Internet such that it accesses the World Wide Web. The digital processing device may be optionally connected to a cloud computing infrastructure. The digital processing device may be optionally connected to an intranet. The digital processing device may be optionally connected to a data storage device.

Suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Many smartphones may be suitable for use in the system described herein. Televisions, video players, and digital music players with optional computer network connectivity may be suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some examples, the operating system may be provided by cloud computing. The device may include a storage and/or memory device. The storage and/or memory device may be one or more physical apparatuses used to store data or programs on a temporary or permanent basis. The device may be volatile memory and may require power to maintain stored information. The device may be non-volatile memory and retains stored information when the digital processing device is not powered. The non-volatile memory may comprise flash memory, dynamic random-access memory (DRAM), ferroelectric random access memory (FRAM), phase-change random access memory (PRAM).

The digital processing device may include a display to send visual information to a user. The display may be a cathode ray tube (CRT), a liquid crystal display (LCD), a thin film transistor liquid crystal display (TFT-LCD), an organic light emitting diode (OLED) display, a passive-matrix OLED (PMOLED) or active-matrix OLED (AMOLED) display, a plasma display, and/or a video projector.

The digital processing device may include an input device to receive information from a user. The input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. The input device may be a Kinect, Leap Motion, or the like. The input device may be a combination of devices such as those disclosed herein.

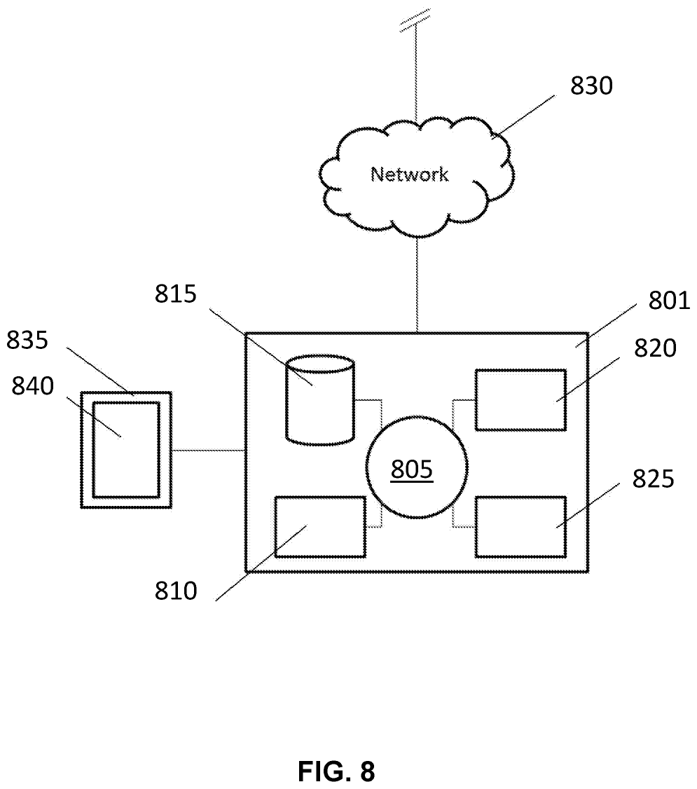

Referring to FIG. 8, an exemplary digital processing device 801 is programmed or otherwise configured to perform annotation or screening. In this example, the digital processing device 801 includes a central processing unit (CPU, also “processor” and “computer processor” herein) 805, which can be a single core or multi core processor, or a plurality of processors for parallel processing. The digital processing device 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The digital processing device 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the device 801, can implement a peer-to-peer network, which may enable devices coupled to the device 801 to behave as a client or a server.

The CPU 805 may execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and write back. The CPU 805 can be part of a circuit, such as an integrated circuit. One or more other components of the device 801 can be included in the circuit. In some cases, the circuit is an application specific integrated circuit (ASIC) or a field programmable gate array (FPGA).

The storage unit 815 may store files, such as drivers, libraries and saved programs. The storage unit 815 can store user data, e.g., user preferences and user programs. The digital processing device 801 in some cases can include one or more additional data storage units that are external, such as located on a remote server that is in communication through an intranet or the Internet.

The digital processing device 801 may communicate with one or more remote computer systems through the network 830. For instance, the device 801 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

Additional Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

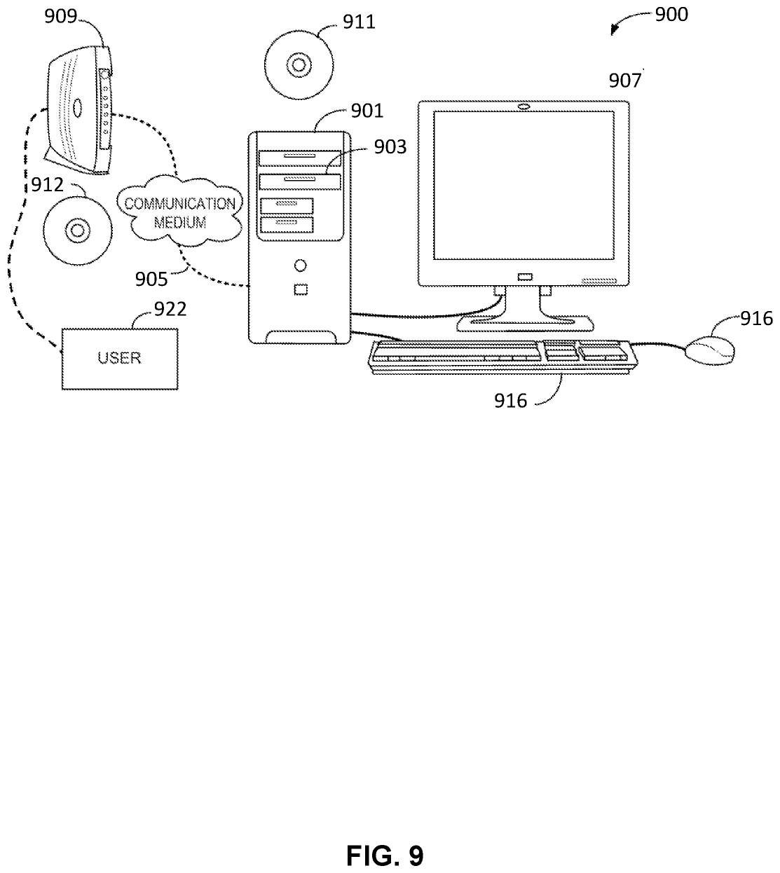

An exemplary computer system 900, as illustrated in FIG. 9, may be understood as a logical apparatus that can read instructions from media 911 and/or a network port 905, which can optionally be connected to server 909 having fixed media 912. The system, such as shown in FIG. 9 can include a CPU 901, disk drives 903, optional input devices such as keyboard 915 and/or mouse 916 and optional monitor 907. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 922 as illustrated in FIG. 9.

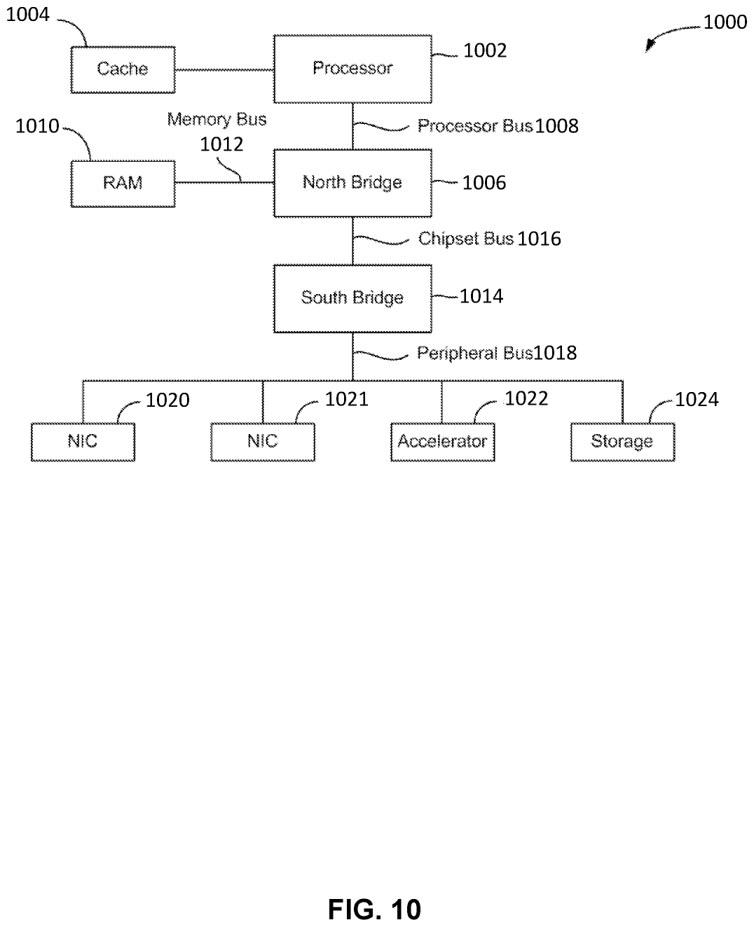

FIG. 10 is a block diagram illustrating a first example architecture of a computer system 1000 that can be used in connection with example instances of the present disclosure. As depicted in FIG. 10, the example computer system can include a processor 1002 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 10, a high speed cache 1004 can be connected to, or incorporated in, the processor 1002 to provide a high speed memory for instructions or data that have been recently, or are frequently, used by processor 1002. The processor 1002 is connected to a north bridge 1006 by a processor bus 1008. The north bridge 1006 is connected to random access memory (RAM) 1010 by a memory bus 1012 and manages access to the RAM 1010 by the processor 1002. The north bridge 1006 is also connected to a south bridge 1014 by a chipset bus 1016. The south bridge 1014 is, in turn, connected to a peripheral bus 1018. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1018. In some alternative architectures, the functionality of the north bridge can be incorporated into the processor instead of using a separate north bridge chip. In some instances, system 1000 can include an accelerator card 1022 attached to the peripheral bus 1018. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1024 and can be loaded into RAM 1010 and/or cache 1004 for use by the processor. The system 1000 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1000 also includes network interface cards (NICs) 1020 and 1021 connected to the peripheral bus for providing network interfaces to external storage, such as Network Attached Storage (NAS) and other computer systems that can be used for distributed parallel processing.

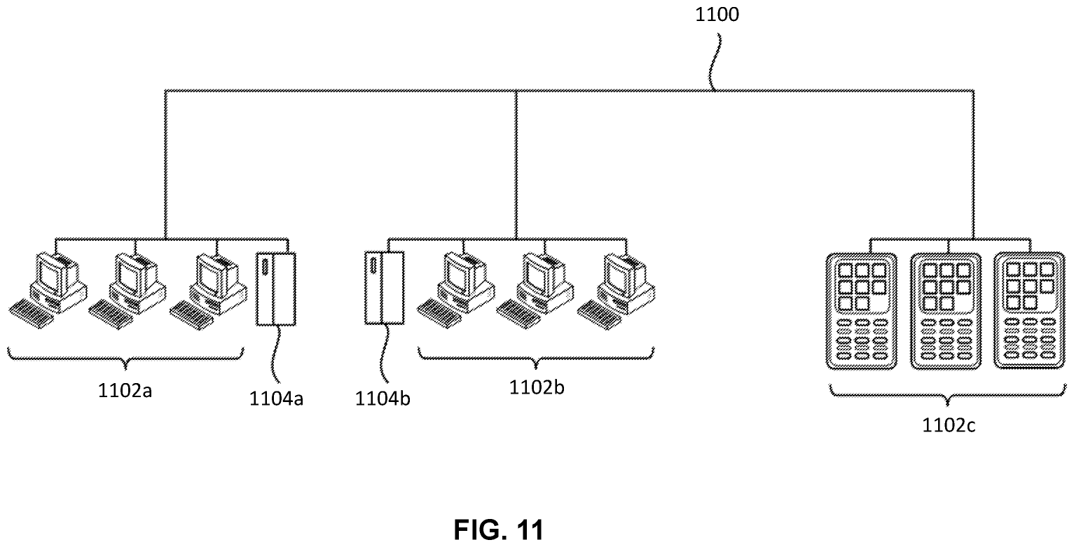

FIG. 11 is a diagram showing a network 1100 with a plurality of computer systems 1102a, and 1102b, a plurality of cell phones and personal data assistants 1102c, and Network Attached Storage (NAS) 1104a, and 1104b. In example instances, systems 1102a, 1102b, and 1102c can manage data storage and optimize data access for data stored in Network Attached Storage (NAS) 1104a and 1104b. A mathematical model can be used for the data and be evaluated using distributed parallel processing across computer systems 1102a, and 1102b, and cell phone and personal data assistant systems 1102c. Computer systems 1102a, and 1102b, and cell phone and personal data assistant systems 1102c can also provide parallel processing for adaptive data restructuring of the data stored in Network Attached Storage (NAS) 1104a and 1104b. FIG. 11 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

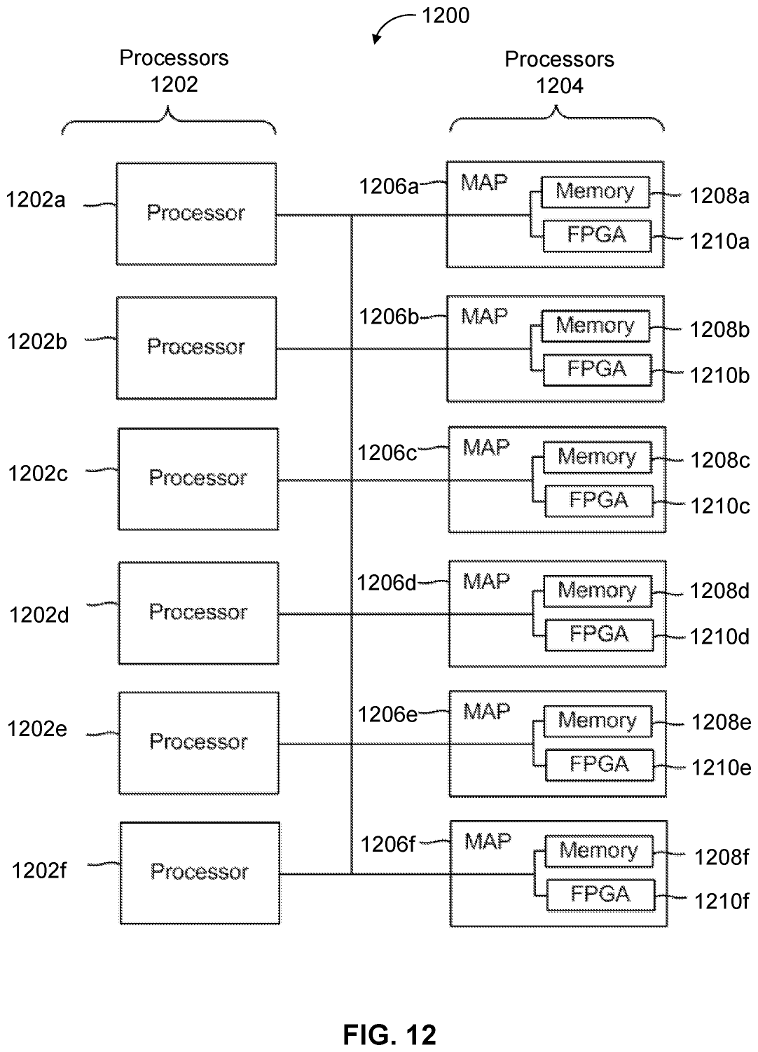

FIG. 12 is a block diagram of a multiprocessor computer system 1200 using a shared virtual address memory space in accordance with an example instance. The system includes a plurality of processors 1202a-f that can access a shared memory subsystem 1204. The system incorporates a plurality of programmable hardware memory algorithm processors (MAPs) 1206a-f in the memory subsystem 1204. Each MAP 1206a-f can comprise a memory 1208a-f and one or more field programmable gate arrays (FPGAs) 1210a-f. The MAP provides a configurable functional unit and particular algorithms or portions of algorithms can be provided to the FPGAs 1210a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1208a-f, allowing it to execute tasks independently of, and asynchronously from the respective microprocessor 1202a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 12, system on chips (SOCs), application specific integrated circuits (ASICs), or other processing and logic elements. For example, the Set Processor and Optimizer can be implemented with hardware acceleration through the use of a hardware accelerator card, such as accelerator card 1022 illustrated in FIG. 10.

Non-Transitory Computer Readable Storage Medium

The platforms, systems, media, and methods disclosed herein may include one or more non-transitory computer readable storage media encoded with a program including instructions executable by the operating system of an optionally networked digital processing device. A computer readable storage medium may be a tangible component of a digital processing device. A computer readable storage medium is optionally removable from a digital processing device. A computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Computer Program

The platforms, systems, media, and methods disclosed herein may include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

Web Application

A computer program described herein may include a web application. A web application may utilize one or more software frameworks and one or more database systems. A web application may be created upon a software framework such as Microsoft .NET or Ruby on Rails (RoR). A web application may utilize one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). A web application may be written to some extent in a presentation definition language such as Cascading Style Sheets (CSS). A web application may be written to some extent in a client-side scripting language such as Asynchronous JavaScript and XML (AJAX), Flash® ActionScript, JavaScript, or Silverlight®. A web application may be written to some extent in a server-side coding language such as Active Server Pages (ASP), ColdFusion®, Perl, Java™, Java Server Pages (JSP), Hypertext Preprocessor (PHP), Python™, Ruby, Tcl, Smalltalk, WebDNA®, or Groovy. A web application may be written to some extent in a database query language such as Structured Query Language (SQL).

Mobile Application

A computer program described herein may include a mobile application provided to a mobile digital processing device. The mobile application may be provided to a mobile digital processing device at the time it is manufactured. The mobile application may be provided to a mobile digital processing device via the computer network described herein.

A mobile application may be created, for example, using hardware, languages, and development environments. Mobile applications may be written in various programming languages. Suitable programming languages include, by way of non-limiting examples, C, C++, C #, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Standalone Application

A computer program described herein may include a standalone application, which is a program that is run as an independent computer process, not an add-on to an existing process, e.g., not a plug-in. Standalone applications may be compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™ Lisp, Python™, Visual Basic, and VB .NET, or combinations thereof. Compilation is often performed, at least in part, to create an executable program.

Web Browser Plug-in

A computer program described herein may include a web browser plug-in. In computing, a plug-in may be one or more software components that add specific functionality to a larger software application. Makers of software applications support plug-ins to enable third-party developers to create abilities which extend an application, to support easily adding new features, and to reduce the size of an application. When supported, plug-ins may enable customizing the functionality of a software application. For example, plug-ins are commonly used in web browsers to play video, generate interactivity, scan for viruses, and display particular file types. Web browser plug-ins include, without limitation, Adobe® Flash® Player, Microsoft® Silverlight®, and Apple® QuickTime®. The toolbar may comprise one or more web browser extensions, add-ins, or add-ons. In some embodiments, the toolbar comprises one or more explorer bars, tool bands, or desk bands.

Several plug-in frameworks may be available that may enable development of plug-ins in various programming languages, including, by way of non-limiting examples, C++, Delphi, Java™, PHP, Python™, and VB .NET, or combinations thereof.

Web browsers (also called Internet browsers) are software applications, which may be configured for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) may be configured for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

Software Modules

The systems, media, networks and methods described herein may include software, server, and/or database modules, or use of the same. Software modules may be created using various machines, software, and programming languages. The software modules disclosed herein are implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. The one or more software modules may comprise, by way of non-limiting examples, a web application, a mobile application, and a standalone application. In some embodiments, software modules are in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

Databases

The platforms, systems, media, and methods disclosed herein may include one or more databases, or use of the same. In view of the disclosure provided herein, many databases are suitable for storage and retrieval of physiological data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. A database may be web-based. A database may be cloud computing-based. A database may be based on one or more local computer storage devices.

Algorithms

The platforms, systems, media, and methods disclosed herein may include one or more algorithms, or use of the same. In view of the disclosure provided herein, many algorithms are suitable for searching and comparing sequence data. In various embodiments, suitable algorithms include, by way of non-limiting examples BLAST, DIAMOND, BLAT, BWT, PLAST, Smith-Waterman, or other algorithm for sequence searching and alignment. Algorithms may include accelerated or extended versions of existing algorithms, or software tools which use these algorithms. In some instances, suitable accelerated or extended algorithms and software tools by way of non-limiting examples include CS-BLAST, Tera-BLAST, GPU-Blast, G-BLASTN, MPIBLAST, Paracel BLAST, CaBLAST, or any other additional algorithms or software tools that accelerate the BLAST algorithm.

It shall be understood that different aspects of the present disclosure can be appreciated individually, collectively, or in combination with each other. The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Assembly Design of a Polynucleotide Greater than 1000 Bases

A full length sequence of 1385 bases in length was inputted into an oligo design algorithm, and iterative runs conducted to identify an optimal design. 10,000 designs were generated for each run, with each run comprising a different set of variables. Length, GC, and RPM (repeating/palindromic motif) filters were initially not used. Multiple runs were conducted with an increasingly tight Tm filter, until no designs were found. The tightest Tm filter that produced at least one design corresponded to a minimum overlap Tm of 59 degrees C. and a maximum overlap Tm of 62 degrees C. Multiple runs were then conducted with the RPM filter on, and runs were repeated with an increasing number of matching bases in the overlap regions until designs passing the RPM filter were found. Using the final parameter set, 36,231 overlaps were created, and 2,267 overlaps were selected after filtering for length, GC, and RPM. A graph of overlaps was generated, and 10,000 paths through the graph were generated and ranked. Each path corresponded to a design, with the highest ranked path represented an optimal design.

### Example 2: Assembly Design of a Full Length Sequence Less than about 2 kb

A full length sequence of less than about 2000 bases in length is inputted into an oligo design algorithm, and iterative runs are conducted to identify an optimal design. A number of designs are generated, in some instances at least 5,000 designs are generated for each run, with each run comprising a different set of variables. Length, GC, and RPM (repeating/palindromic motif) filters are initially not used. Multiple runs are conducted with an increasingly tight Tm filter, until no designs are found. The tightest Tm filter that produced at least one design is used for further optimization with filters. Multiple runs are then conducted with the RPM filter on, and runs are repeated with an increasing number of matching bases in the overlap regions until designs passing the RPM filter are found. Using the final parameter set, at least 30,000 overlaps are created, and at least 1,000 overlaps are selected after filtering for length, GC, and RPM. A graph of overlaps is generated, and paths through the graph are generated and ranked, in some instances at least 5,000 paths. Each path corresponds to a design, with the highest ranked path corresponding to an optimal design.

### Example 3: Split-Point Optimization

A full length sequence greater than 2 kb in length is inputted into the oligo design algorithm, and the sequence is divided into a first sub-sequence and a second sub-sequence. The split point is initially determined by dividing the full length sequence so that the first and second sub-sequences are about equal length. The split point is then varied in both directions for a predetermined number of bases, to maximize disruption of local repeat sequences, and distribute repeats across the two subsequences. Once an optimal split point is established, the splitting process is repeated for each sub-sequence until fragments of a desired maximum length are generated, including an overlap region between fragments. The sub-sequences are then individually subjected to design generation using the general methods of Example 1.

### Example 4: Scoring

Designs are generated using the general procedure of Example 3, with the modification that an initial value is set for the maximum fragment length. The full length sequence is then divided into sub-sequences using this maximum fragment length, and each fragment is subjected to the assembly design algorithm. Additionally, direct and inverted repeats are annotated on the full length sequence, to aid in identifying complex sequences.

### Example 5: Automated Polynucleotide Synthesis

A full length sequence is inputted into an oligo design algorithm, and an optimal design is generated using the general methods of Examples 1-4. The full length polynucleotide is automatically synthesized via synthesis of all of the fragment sequences, and assembly of the fragment sequences with PCR using fragment sequences and conditions obtained from the highest ranked design. Optionally, the synthesized full length polynucleotide is sequenced for accuracy, and shipped. In some instances, sequencing and shipping processes are automated.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### Example 6: Assembly Design and Selection

A plurality of designs are generated for a full length sequence of 5 kb using the general methods of Examples 1 and 2 with modification. Overlap lengths are restricted to 30-50 bases, and a design is selected that (a) had the lowest variance in Tm across all overlaps and (b) does not have any overlaps comprising homopolymeric sequences. The selected fragments from this design are then synthesized, assembled by PCA, ligated into a vector, and transformed into a host organism, such as E. coli. After plating the transformed organism cells onto agar, colonies are picked from the plate, cultured, the vectors extracted, and subjected to sequencing to identify correctly assembled full length sequences.

### Example 7: Polynucleotide Scoring

Polynucleotides from a data set comprising 86,929 sequences were each scored using weighted categories (or features): average percent GC content of the sequence; the percent GC content for a region of continuous bases in the sequence; synthesis sequence length; maximum melting temperature for direct repeats in the sequence; density of repeats in the sequence; and length of homopolymers in the sequence. For example, the lowest scores were assigned to sequences comprising an overall GC percent of 25-60%, windowed % GC content of 10-50%, a length of less than 1700 bp, a direct repeat max Tm of less than 57 degrees C., a repeat density of less than 0.1, and homopolymer or multimer lengths of less than 20 bases. Scores obtained for each of the sequences was then plotted against the percentage of the corresponding correctly assembled polynucleotides after synthesis and assembly (FIG. 7). Higher pass rates were well-correlated with a lower score.

### Example 8: Adjusting Clonal Sampling with Polynucleotide Scoring

A full length sequence is scored using the general method of Example 7, and then a design is selected, fragments synthesized, and fragments assembled using the general methods of Example 6, with modification. Based on the score obtained, the number of colonies sampled from the host organism either increases or decreases to reflect the difficulty or ease of the assembly, respectively. For example, a design receiving a low score requires fewer colonies sampled (such as 4 or fewer), as there is a higher likelihood that a colony will comprise the correctly assembled full length polynucleotide. A design receiving a higher score requires a larger number of colonies to be sampled (for example, at least 8, or at least 24) to identify a colony comprising the correctly assembled full length polynucleotide.

### Example 9: Split-Point Optimization

A full length sequence greater than 2 kb in length is inputted into the oligo design algorithm, and the sequence is divided into sub-sequences using the general methods of example 3, with modification. Split points are established using gradient descent or genetic algorithm-based methods. The sub-sequences are then individually subjected to design generation using the general methods of Example 1.

What is claimed is:

1. A computerized system for polynucleotide assembly comprising:
a general purpose computer; and
a computer readable medium comprising functional modules including instructions for the general purpose computer, wherein said computerized system is configured for operating in a method of:
i) receiving operating instructions, wherein the operating instructions comprise a full length polynucleotide sequence;
ii) automatically generating a plurality of designs each comprising a plurality of polynucleotide sequences, wherein the plurality of polynucleotide sequences each comprises at least one overlap region of 30 to 50 bases in length, wherein each overlap region is complementary to another overlap region, and wherein each of the at least one overlap regions does not comprise a homopolymeric sequence; and
iii) automatically selecting a design from the plurality of designs that comprises polynucleotide sequences having the lowest variance in Tm between the at least one overlap regions.

2. The computerized system of claim 1, wherein assembly of the polynucleotide sequences having the lowest variance in Tm between the at least one overlap regions results in the full length polynucleotide sequence.

3. The computerized system of claim 1 or 2, wherein the full length polynucleotide sequence is at least 500 bases in length.

4. The computerized system of any one of claims 1-3, wherein the full length polynucleotide sequence is at least 2,000 bases in length.

5. The computerized system of any one of claims 1-4, wherein the full length polynucleotide sequence is at least 5,000 bases in length.

6. The computerized system of any one of claims 1-5, wherein the full length polynucleotide sequence is at least 10,000 bases in length.

7. The computerized system of claim 1, wherein the full length polynucleotide sequence is at least 1,000 bases in length.

8. The computerized system of any one of claims 1-7, wherein the at least one overlap regions comprises an average of 30 percent to 70 percent GC content.

9. The computerized system of claim 1, wherein the at least one overlap regions comprises an average of 40 percent to 60 percent GC content.

10. The computerized system of any one of claims 1-9, wherein each of the at least one overlap regions comprises 30 percent to 70 percent GC content.

11. The computerized system of claim 1, wherein each of the at least one overlap regions comprises 40 percent to 70 percent GC content.

12. The computerized system of any one of claims 1-11, wherein each of the at least one overlap regions is 20 to 40 bases in length.

13. The computerized system of claim 1, wherein each of the at least one overlap regions is 25 to 40 bases in length.

14. The computerized system of any one of claims 1-13, wherein the plurality of polynucleotide sequences comprises at least 5 polynucleotide sequences.

15. The computerized system of any one of claims 1-14, wherein the plurality of polynucleotide sequences comprises at least 50 polynucleotide sequences.

16. The computerized system of claim 1, wherein the plurality of polynucleotide sequences comprises at least 10 polynucleotide sequences.

17. The computerized system of any one of claims 1-13, wherein the plurality of polynucleotide sequences comprises 25 to 50 polynucleotide sequences.

18. The computerized system of claim 1, wherein the plurality of polynucleotide sequences comprises 10 to 30 polynucleotide sequences.

19. The computerized system of any one of claims 1-18, wherein each polynucleotide sequence is 40 to 200 bases in length.

20. The computerized system of claim 1, wherein each polynucleotide sequence is 50 to 150 bases in length.

21. The computerized system of any one of claims 1-20, wherein the full length polynucleotide sequence encodes a cDNA sequence for a gene or gene fragment.

22. A method for polynucleotide synthesis comprising:
a) receiving operating instructions, wherein the operating instructions comprise a full length polynucleotide sequence;
b) automatically generating a plurality of designs each comprising a plurality of polynucleotide sequences, wherein the plurality of polynucleotide sequences each comprises at least one overlap region of 30 to 50 bases in length, wherein each overlap region is complementary to another overlap region, and wherein each of the at least one overlap regions does not comprise a homopolymeric sequence;
c) automatically selecting a design from the plurality of designs that comprises polynucleotide sequences having the lowest variance in Tm between the at least one overlap regions; and
d) synthesizing the polynucleotide sequences having the lowest variance in Tm between the at least one overlap regions.

23. The method of claim 22, further comprising assembling the full length polynucleotide sequence from the polynucleotide sequences having the lowest variance in Tm between the at least one overlap regions.

24. The method of any one of claims 22-23, wherein the full length polynucleotide sequence is at least 500 bases in length.

25. The method of any one of claims 22-24, wherein the full length polynucleotide sequence is at least 5,000 bases in length.

26. The method of claim 22, wherein the full length polynucleotide sequence is at least 1,000 bases in length.

27. The method of any one of claims 22-26, wherein the at least one overlap regions comprise an average of 30 percent to 70 percent GC content.

28. The method of claim 22, wherein the at least one overlap regions comprise an average of 40 percent to 60 percent GC content.

29. The method of any one of claims 22-26, wherein in each of the at least one overlap regions comprises 30 percent to 70 percent GC content.

30. The method of claim 22, wherein in each of the at least one overlap regions comprises 40 percent to 60 percent GC content.

31. The method of any one of claims 22-30, wherein each of the at least one overlap regions is 20 to 40 bases in length.

32. The method of claim 22, wherein each of the at least one overlap regions is 25 to 40 bases in length.

33. The method of any one of claims 22-27, wherein the plurality of polynucleotide sequences comprises at least 5 polynucleotide sequences.

34. The method of any one of claims 22-28, wherein the plurality of polynucleotide sequences comprises at least 50 polynucleotides sequences.

35. The method of claim 22, wherein the plurality of polynucleotide sequences comprises at least 10 polynucleotide sequences.

36. The method of any one of claims 22-35, wherein each polynucleotide sequence is 40 to 200 bases in length.

37. The method of claim 22, wherein each polynucleotide sequence is 50 to 150 bases in length.

38. The method of any one of claims 22-37, wherein the full length polynucleotide sequence encodes a cDNA sequence for a gene or gene fragment.

39. A computerized system for polynucleotide assembly comprising:
a general purpose computer; and
a computer readable medium comprising functional modules including instructions for the general purpose computer, wherein said computerized system is configured for operating in a method of:
a) receiving operating instructions, wherein the operating instructions comprise a full length polynucleotide sequence;
b) automatically generating a plurality of designs each comprising a plurality of polynucleotide sequences;
c) automatically generating a pass score for each of the polynucleotide sequences, wherein the pass rate score is determined by assigning a weighted value for one or more of:
i. average percent GC content of the polynucleotide sequence;
ii. the percent GC content for a region of continuous bases in the polynucleotide sequence;
iii. length of the polynucleotide sequence;
iv. maximum melting temperature for direct repeats in the polynucleotide sequence;
v. length of direct repeats;
vi. density of repeats in the polynucleotide sequence, wherein the density of repeats is a number of repeating bases divided by a total length of each polynucleotide sequence; and
vii. length of homopolymers in the polynucleotide sequence; and
d) assigning a numerical value to at least one design for a number of clones to screen for the full length sequence following assembly, wherein the numerical value is assigned based on the pass rate score.

40. The computerized system of claim 39, wherein the pass rate score is determined by assigning a weighted value to the percent GC content for a region of continuous bases in the polynucleotide sequence, and wherein the region of continuous bases in the polynucleotide sequence is at least 25 bases in length.

41. The computerized system of claim 39 or 40, wherein the number of repeating bases is at least 6 bases.

42. The computerized system of claim 39, wherein the number of repeating bases is 6-15 bases.

43. The computerized system of any one of claims 39-42, wherein the homopolymers each have a length of at least 10 bases.

44. The computerized system of claim 39, wherein the homopolymers each have a length of 6-15 bases.

45. The computerized system of any one of claims 39-44, wherein the plurality of polynucleotide sequences comprises at least 30 polynucleotide sequences.

46. The computerized system of claim 39, wherein the plurality of polynucleotide sequences comprises 25-50 polynucleotide sequences.

47. The computerized system of any one of claims 39-46, wherein the clones are generated by prokaryotic cells or eukaryotic cells.

48. The computerized system of any one of claims 39-47, wherein the method further comprises rejecting a design that receives a numerical value less than a predetermined numerical value threshold, and wherein nucleic acids encoding for the polynucleotide sequences of the rejected design are not synthesized.

49. The computerized system of any one of claims 39-48, wherein the method further comprises synthesizing nucleic acids encoding for the plurality of polynucleotide sequences from at least one design.

50. The computerized system of claim 49, wherein the method further comprises assembling the plurality of polynucleotides of at least one design into a nucleic acid encoding for the full length polynucleotide sequence, wherein assembling comprising PCA.

51. The computerized system of claim 50, wherein the method further comprises transforming the nucleic acid encoding for the full-length polynucleotide sequence into at least one cell to generate at least one clone.

52. The computerized system of claim 51, wherein the method further comprises sequencing at least one clone to confirm assembly of the nucleic acid encoding for the full length polynucleotide sequence.

53. A method for polynucleotide synthesis comprising:
a) receiving operating instructions, wherein the operating instructions comprise a full length polynucleotide sequence;
b) automatically generating a plurality of designs each comprising a plurality of polynucleotide sequences;
c) automatically generating a pass score for each of the polynucleotide sequences, wherein the pass rate score is determined by assigning a weighted value for one or more of:
i. average percent GC content of the polynucleotide sequence;
ii. the percent GC content for a region of continuous bases in the polynucleotide sequence;
iii. length of the polynucleotide sequence;
iv. maximum melting temperature for direct repeats in the polynucleotide sequence;
v. length of direct repeats;
vi. density of repeats in the polynucleotide sequence, wherein the density of repeats is a number of repeating bases divided by a total length of the polynucleotide sequence; and
vii. length of homopolymers in the polynucleotide sequence;
d) assigning a numerical value to at least one design for a number of clones to screen for the full length sequence following assembly, wherein the numerical value is assigned based on the pass rate score; and
e) synthesizing polynucleotides having the pass score above a threshold value.

54. The method of claim 53, further comprising assembling the full length polynucleotide sequence from the polynucleotides having the pass score above a threshold value.

55. The method of claim 53, wherein the pass rate score is determined by assigning a weighted value to the percent GC content for a region of continuous bases in the polynucleotide sequence, and wherein the region of continuous bases in the polynucleotide sequence is at least 25 bases in length.

56. The method of any one of claims 53-55, wherein the number of repeating bases is at least 6 bases.

57. The method of claim 53, wherein the number of repeating bases is 6-15 bases.

58. The method of any one of claims 53-57, wherein the homopolymers each have a length of at least 10 bases.

59. The method of claim 53, wherein the homopolymers each have a length of 6-15 bases.

60. The method of any one of claims 53-59, wherein the plurality of polynucleotide sequences comprises at least 30 polynucleotide sequences.

61. The method of claim 53, wherein the plurality of polynucleotide sequences comprises 25-50 polynucleotide sequences.

62. The method of any one of claims 53-61, wherein the clones are generated by prokaryotic cells or eukaryotic cells.

63. The method of any one of claims 53-62, wherein the method further comprises rejecting a design that receives a numerical value less than a predetermined numerical value threshold, and wherein nucleic acids encoding for the polynucleotide sequences of the rejected design are not synthesized.

64. The method of any one of claims 53-63, wherein the method further comprises synthesizing nucleic acids encoding for the plurality of polynucleotide sequences from at least one design.

65. The method of claim 64, wherein the method further comprises assembling the plurality of polynucleotides of at least one design into a nucleic acid encoding for the full length polynucleotide sequence, wherein assembling comprising PCA.

66. The method of claim 65, wherein the method further comprises transforming the nucleic acid encoding for the full-length polynucleotide sequence into at least one cell to generate at least one clone.

67. The method of claim 66, wherein the method further comprises sequencing at least one clone to confirm assembly of the nucleic acid encoding for the full length polynucleotide sequence.


SEQUENCE LISTING (3 sequences)

SEQ ID NO: 1 - DNA, 52 bp, Artificial Sequence
  Description of Artificial Sequence Synthetic oligonucleotide
agtgtgctgacggtgctcggggggggggggggttcgatagctacgatcagct

SEQ ID NO: 2 - DNA, 14 bp, Artificial Sequence
  Description of Artificial Sequence Synthetic oligonucleotide
accgtcagcacact

SEQ ID NO: 3 - DNA, 18 bp, Artificial Sequence
  Description of Artificial Sequence Synthetic oligonucleotide
tgctgatcgtagctatcg